(19) United States
(12) Patent Application Publication
(10) Pub. No.: US 2025/0077879 A1
(43) Pub. Date: Mar. 6, 2025

(54) METHOD AND APPARATUS FOR RECOGNIZING EMOTION USING BRAIN SIGNAL DATA

(71) Applicant: ELECTRONICS AND TELECOMMUNICATIONS RESEARCH INSTITUTE, Daejeon (KR)

(72) Inventors: Zubair MUHAMMAD, Daejeon (KR); Sungpil WOO, Daejeon (KR); Sunhwan LIM, Daejeon (KR)

(21) Appl. No.: 18/524,382

(22) Filed: Nov. 30, 2023

(30) Foreign Application Priority Data
Sep. 5, 2023 (KR) 10-2023-0117667

(51) Int. Cl. G06N 3/091; G06F 18/2415

(52) U.S. Cl. CPC G06N 3/091; G06F 18/2415

(57) ABSTRACT

A method of recognizing an emotion using brain signal data includes a step in which an emotion recognition apparatus receives brain signal data, a step in which the emotion recognition apparatus inputs the brain signal data into an emotion analysis model, and a step in which the emotion recognition apparatus recognizes an emotion on the basis of an output value of the emotion analysis model. The emotion analysis model may be a model configured to extract a domain-invariant and emotion-specific feature and then analyze an emotion on the basis of the extracted feature.

### CROSS REFERENCE TO RELATED APPLICATION

The present application claims priority to Korean Patent Applications No. 10-2023-0117667, filed Sep. 5, 2023 the entire contents of which are incorporated herein for all purposes by this reference.

### BACKGROUND

### 1. Technical Field

The technology to be described hereafter provides a method of recognizing an emotion using brain signal data.

### 2. Description of Related Art

An emotion recognition technology is a technology of recognizing emotions of a human on the basis of biological signals. An emotion recognition technology has used biological signals such as electrocardiogram (ECG), electromyogram (EMG), and electroencephalogram (EEG). Recently, an emotion recognition technology using deep learning is under development with the start of development of the deep learning technology that uses an artificial neural network. For example, emotions have been recognized by collecting EGG data, constructing learning data by labeling correct answers to the collected data, constructing an emotion analysis model on the basis of the constructed learning data, and using the emotion analysis model. An emotion analysis model constructed in this way has been received EGG data and has output emotion recognition results.

### PRIOR ART DOCUMENT

(Patent Document 1) U.S. Patent Application Publication No. 2023-0039900 A

### SUMMARY

A domain shift problem may be generated in the process of constructing a learning model such as deep learning. A domain shift usually appears when using data obtained from different subjects. A domain shift means a phenomenon where a model trained using learning data of one domain cannot analyze well the data at another domain. For example, it means a phenomenon where a learning model trained using brain signal data measured by an apparatus A cannot infer using brain signal data measured by an apparatus B.

A larger domain shift may appear in models that use brain signal data. This is because the variability of brain signal data depending on an atmosphere, time, people, etc. is large. Accordingly, a deep learning model trained on the basis of brain signal data measured from people in one group may not analyze well the brain signal data of people not included in learning data.

In order to solve the problems described above, the technology to be described below provides an emotion recognition method and an emotion analysis model construction method that use brain signal data.

A method of recognizing an emotion using brain signal data includes: a step in which an emotion recognition apparatus receives brain signal data; a step in which the emotion recognition apparatus inputs the brain signal data into an emotion analysis model; and a step in which the emotion recognition apparatus recognizes an emotion on the basis of an output value of the emotion analysis model.

The emotion analysis model may be a model configured to extract a domain-invariant and emotion-specific feature and then analyze an emotion on the basis of the extracted feature.

A method of constructing an emotion analysis model includes: (a) a step in which the learning apparatus receives source data and target data; (b) a step in which the learning apparatus extracts a feature of each of data by inputting the source data and the target data into a feature extractor module of the emotion analysis model; (c) a step in which the learning apparatus computes a result of expecting emotion recognition by inputting a feature extracted from the source data into a classifier module of the emotion analysis model; (d) a step in which the learning apparatus inputs a feature extracted from the target data and a feature extracted from the source data into a domain discriminator module of the emotion analysis model and then computes the degree of discrimination by the domain discriminator module; and (e) a step in which the learning apparatus updates parameters of the emotion analysis model such that accuracy of the result of expecting the emotion recognition is improved and an error of the degree of discrimination by the domain discriminator module is increased.

The source data and the target data may be brain signal data belonging to different domains and the source data may include an emotion recognition result that is reference data.

Using the technology to be described below makes it possible to recognize emotions on the basis of brain signal data. In detail, it is possible to determine the levels of valence and arousal on the basis of brain waves by using the technology to be described below.

Using the technology to be described below makes it possible to extract domain-invariant features when extracting features from brain signal data. Accordingly, even though data of a domain not included in learning data is input, a model can recognize an emotion well. Therefore, it is possible to minimize a domain shift problem.

By using the technology to be described below, it is possible to recognize emotions of a human using deep representation determining the inter-relationship between brain wave channels. When the technology to be described below is used, it is possible to improve the performance of a model by further determining emotion-specific bands and channels in brain signal data.

### BRIEF DESCRIPTION OF THE DRAWINGS

The above and other objectives, features and other advantages of the present disclosure will be more clearly understood from the following detailed description when taken in conjunction with the accompanying drawings, in which:

FIG. 1 shows an entire process in which an emotion recognition apparatus recognizes emotions using brain signal data;

FIG. 2 is a flowchart of an embodiment of a method of recognizing an emotion using brain signal data;

FIG. 3 is one of embodiments of an emotion analysis model;

FIG. 4 is one of embodiments of an attention mechanism;

FIG. 5 is a flowchart of an embodiment of a process in which a learning apparatus constructs an emotion analysis model; and

FIG. 6 is one of embodiments of constructing an emotion analysis model using a domain discriminator module; and

FIG. 7 is one configuration of embodiments of an emotion recognition apparatus.

### DETAILED DESCRIPTION

The technology to be described hereafter may be changed in various ways and may have various embodiments. Specific embodiments of the technology to be described hereafter may be shown in the figures of the specification. However, this is for explaining the technology to be described hereafter without intending to limit the technology to be described hereafter to specific embodiments. Accordingly, it should be understood that all of modifications, equivalents, and substitutions that are included in the spirit and scope of the technology to be described hereafter are included in the technology to be described hereafter.

Terms such as first, second, A, B, etc. may be used to describe various embodiments. However, these terms are used only to discriminate one component from other components without intending to limit corresponding components. For example, the first component may be named the second component, and vice versa, without departing from the scope of the technology to be described hereafter. The term “and/or” includes a combination of a plurality of relevant items or any one of a plurality of relevant terms.

In the terms to be used hereafter, singular forms should be understood as including plural forms unless the context clearly indicates otherwise and it will be further understood that the terms “comprises” or the like used in this specification specify the presence of stated features, numbers of articles, steps, operations, components, parts, or a combination thereof, but do not preclude the presence or addition of one or more other features, numbers of articles, steps, operations, components, parts, or a combination thereof.

Before describing the drawings in detail, it should be clearly understood that components described herein are discriminated on the basis of their main functions.

That is, two or more components to be described below may be combined into one component or one component may be divided into two or more for more detailed functions. Each of components to be described hereafter may additionally perform some or all of the function of another component in addition to its main function, and some functions of the main function of each component may be performed exclusively by another component.

When performing a method or an operation method, the processes constituting the method may occur in different order from the described order unless a specific order is clearly stated. That is, each process may occur in the order described herein, may be substantially simultaneously performed, or may be performed in a reverse order.

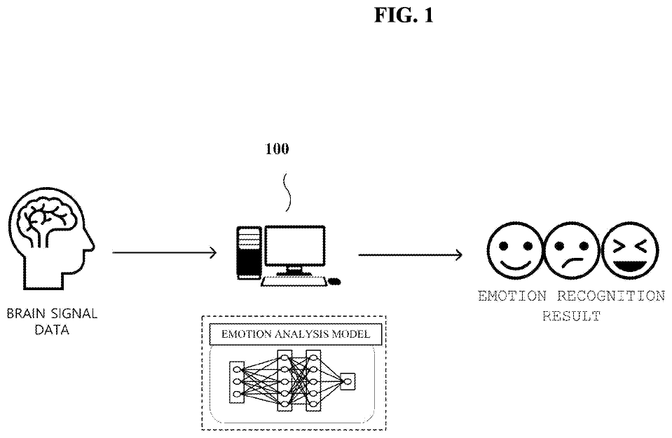

Hereafter, the entire process of a method in which an emotion recognition apparatus recognizes emotions using brain signal data is described through FIG. 1.

FIG. 1 shows an entire process in which an emotion recognition apparatus 100 recognizes emotions using brain signal data.

The emotion recognition apparatus 100 can receive brain signal data. The emotion recognition apparatus 100 can input brain signal data into an emotion analysis model. The emotion recognition apparatus 100 can recognize emotions on the basis of output values of the emotion analysis model.

The emotion recognition model can extract domain-invariant and emotion-specific features. The emotion analysis model may be a model that analyzes emotions on the basis of the extracted features.

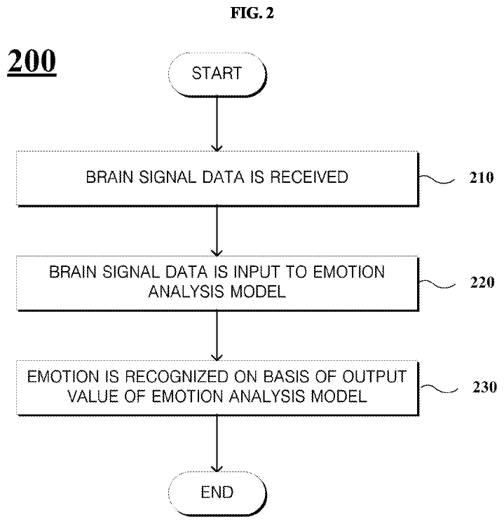

Hereafter, a method of recognizing an emotion using brain signal data is described in detail through FIG. 2.

FIG. 2 is a flowchart 200 of an embodiment of a method of recognizing an emotion using brain signal data.

The emotion recognition apparatus 100 can receive brain signal data 210.

The brain signal data may mean data measuring the brain waves of a subject. The brain signal data may be Electroencephalography (EEG) signals. The brain data may be data measuring electrical activity of a brain through electrodes.

The emotion recognition apparatus can input brain signal data into an emotion analysis model in step 220.

The emotion analysis model may be a model that analyzes what the current emotion of a person is on the basis of brain signal data.

The emotion analysis model may be a learning model trained to analyze emotions of a human using learning data. As an embodiment, the emotion analysis model may be a machine learning-based model. In more detail, the emotion analysis model may be a model based on an Artificial Neural Network (ANN).

The emotion analysis model may be a classification model. For example, the emotion analysis model may be a model that classifies brain signal data into brain signal data when a human feels good or brain signal data when a human feels bad. Alternatively, the emotion analysis model may be a model that classifies brain signal data into brain signal data when a human is aroused or brain signal data when a human is not aroused.

The emotion analysis model may include a feature extractor module and a classifier module. The feature extractor module may be a module that extracts domain-invariant and emotion-specific features from input brain signal data. The classifier module may be a module that classifies the emotions of a subject when brain signal data is measured on the basis of the features extracted by the feature extractor module. The detailed architecture of the emotion analysis model will be described below.

The emotion analysis model may be a model trained to minimize a domain shift problem. To this end, the emotion analysis model may use a domain discriminator module in a learning process. The detailed constructing (learning) process of the emotion analysis model will be described below.

The emotion recognition apparatus can recognize emotions on the basis of output values of the emotion analysis model.

As an embodiment, the emotion recognition apparatus can recognize the arousal level and the valence level of a subject on the basis of output values of the emotion analysis model. Alternatively, the emotion recognition apparatus can recognize whether a human is happy, depressed, tired, etc. on the basis of output values of the emotion analysis model.

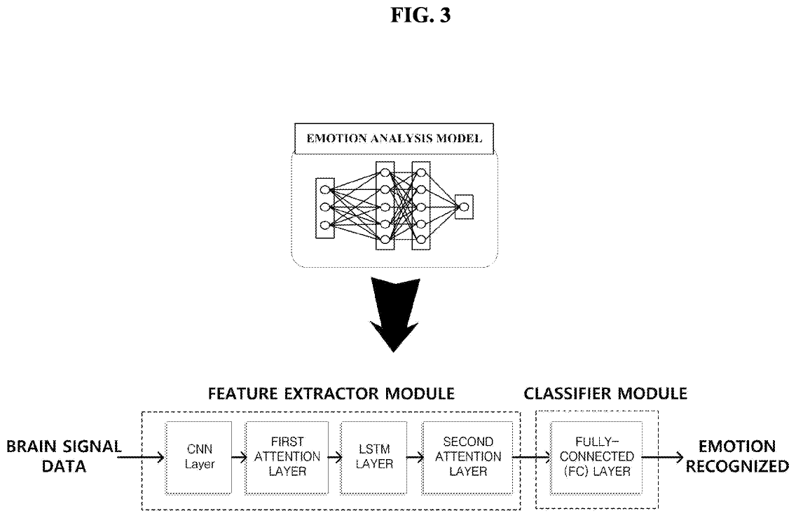

Hereafter, the architecture of the emotion analysis model is described in detail through FIG. 3.

FIG. 3 is one of embodiments of an emotion analysis model.

The emotion analysis model may include a feature extractor module and a classifier module.

The feature extractor module may be a module that extracts features from input brain signal data. As will be described below, the features that the feature extractor module extracts may be domain-invariant and emotion-specific features.

The feature extractor module may sequentially include a Convolution neural Network (CNN) layer, a first attention layer, a Long-short Term Memory (LSTM) layer, and a second attention layer.

The CNN layer may be a layer that performs convolution. The CNN layer can extract features that represent bands by determining the inter-relationship between different bands of brain wave measurement channels from brain signal data.

The first attention layer may be a layer that performs an attention mechanism. The first attention layer can receive an output value of the CNN layers. The first attention layer can emphasize a feature for a band having high relevance to emotion recognition of extracted features. That is, an output of the first attention layer may be a value obtained emphasizing a band having high relevance to emotion recognition of bands of brain wave measurement channels.

The LSTM layer may be a layer that performs long-short term memory. The LSTM layer can receive an output value of the first attention layer. The LSTM layer may be a layer for determining the inter-relationship between channels to efficiently extract features.

The second attention layer may be a layer that performs an attention mechanism, similar to the first attention layer. The second attention layer can receive an LSTM output value. The second attention layer can emphasize a channel having high relevance to emotion recognition of results output by the LSTM layer. That is, an output value of the second attention layer may be a value obtained by emphasizing a channel having high relevance to recognizing emotions in brain wave measurement channels. In short, the first attention layer emphasizes a band having high relevance to recognizing emotions from brain signal data and the second attention layer emphasizes a channel band having high relevance to recognizing emotions.

As will be described below, in the learning process of the emotion analysis model, the first attention layer and the second attention layer may compute features to be input to the classifier module, but may compute peripheral features that are features irrelevant to emotion recognition. Details will be described in the learning process of the emotion analysis model.

The classifier module can receive features extracted by the feature extractor module. The classifier module can classify emotions of a subject when brain signal data is measured on the basis of the input features.

The classifier module may include two fully connected layers and two softmax layers.

The classifier module can output three values.

The first one is value related to an arousal level. The arousal level may mean the degree of agitation of emotion. A low arousal level shows that it is calm and a high arousal level shows that it is agitated.

The second one relates to a valence level. The valence level means the degree of positive or negative emotion. A low valence level shows that it is negative and a high valence level shows that it is positive.

The third one relates to a domain similarity score. The domain similarity score may be used to train the emotion analysis model. Details will be described in the learning process of the emotion analysis model.

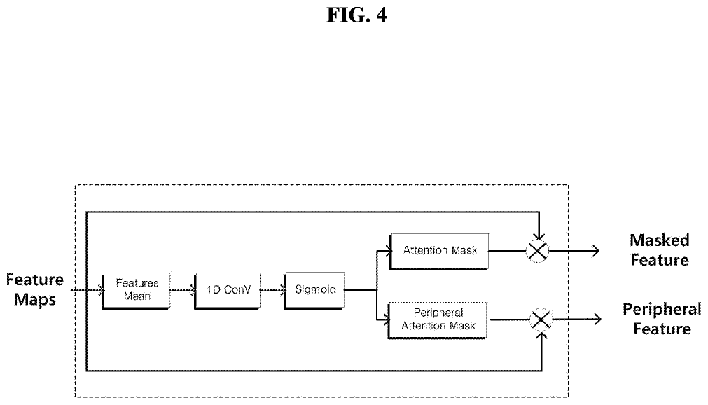

Hereafter, the attention mechanism that is performed by the first attention layer and the second attention layer is described in detail through FIG. 4.

FIG. 4 is one of embodiments of an attention mechanism.

An attention mechanism is one of operations emphasizing values having a great influence of an analysis result in input values.

First, a mean is computed along a channel axis in a feature map that is input. Thereafter, the first attention layer computes an attention score through a 1D convolution layer and a sigmoid layer. It is possible to create an attention mask on the basis of the computed attention score. The first attention layer can emphasize some of input features on the basis of the attention mask.

The second attention array also performs an attention mechanism in the similar way to the first attention array. However, the second attention array is different from the first attention array in that the second attention array computes a mean long a band axis in a feature map. That is, the first attention array emphasizes a band having a great influence on an output value in an input feature map and the second attention array emphasizes a channel having a great influence on an output value in an input feature map.

The attention mask may be used also to compute a peripheral feature. As an embodiment, it is possible to form a peripheral attention mask by inverting an attention mask and then using the peripheral attention mask.

A peripheral feature means a feature that has a small influence on a result value. Further, the peripheral feature may be a feature that generally is shown in brain signal data of many people.

The peripheral feature can promote a domain discriminator module to be able to discriminate the domain of brain signal data. Further, the peripheral feature can make a domain extractor module output invariant features to a domain.

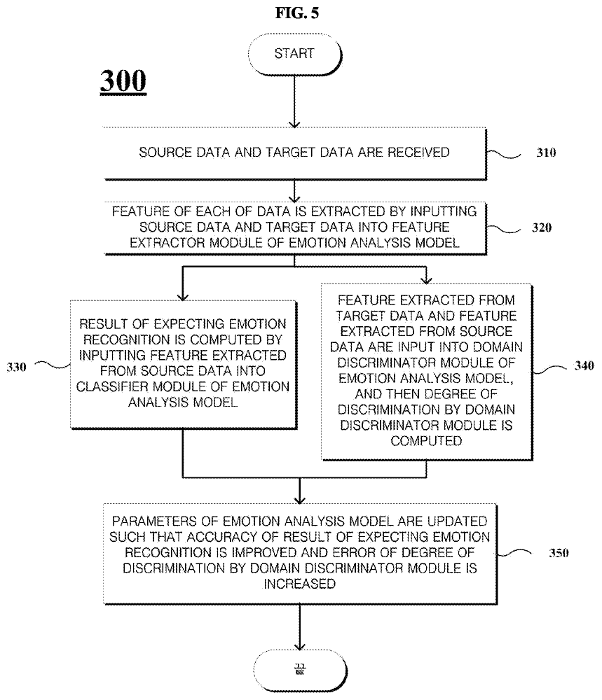

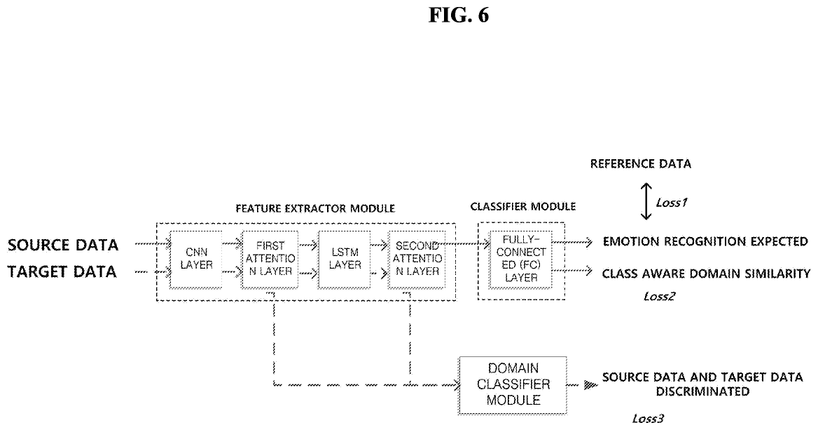

Hereafter, a process of constructing an emotion analysis model is described in detail through FIGS. 5 and 6.

FIG. 5 is a flowchart 300 of an embodiment of a process in which a learning apparatus constructs an emotion analysis model. FIG. 6 is one of embodiments of constructing an emotion analysis model using a domain discriminator module.

The process of constructing an emotion analysis model can be performed by a learning apparatus. The learning apparatus may be an apparatus that is separate from or the same as an emotion analysis apparatus. In other words, the emotion analysis apparatus can receive an emotion analysis model constructed by a separate apparatus and perform an emotion analysis method or can construct an emotion analysis model by itself and perform an emotion analysis method. It is exemplified hereafter for the convenience of description that a learning apparatus constructs an emotion analysis model.

A learning apparatus can receive source data and target data in step 310.

The source data and the target data may be brain signal data belonging to different domains. For example, when source data is brain signal data measured using an apparatus A, target data may be brain signal data measured using an apparatus B. Alternatively, when source data is brain signal data measured from Koreans, target data may be brain signal data measured from Chinese.

Source data is used to train the feature extractor module and the classifier module. In detail, source data may be used to train the feature extractor module to extract features that can best represent brain signal data. Alternatively, source data may be used to train the classifier module to recognize emotions of a human on the basis of extracted features. Accordingly, source data may include an emotion recognition result that is reference data labeled with correct answers.

Target data is used to train parameters of the feature extractor module and the domain discriminator module. In detail, target data is used to train the feature extractor module to extract domain-invariant and emotion-specific features.

The learning apparatus can extract the feature of each of data by inputting the source data and the target data into the feature extractor module of the emotion analysis model in step 320.

Accordingly, the learning apparatus can extract a feature for the source data and a feature for the target data.

The learning apparatus can compute a result of expecting emotion recognition by inputting the feature extracted from the source data into the classifier module of the emotion analysis model in step 330.

As described above, since labeled reference data is included in the source data, the learning apparatus can compute accuracy of the expectation result by comparing an emotion recognition result expected from the source data and the labeled reference data.

The learning apparatus can input the feature extracted from the target data and the feature extracted from the source data into the domain discriminator module of the emotion analysis model and can compute the degree of discriminating the target data and the source data of the domain discriminator module in step 340.

The domain discriminator module may be a module that can discriminate which one of the source data and the target data the input data that is input comes from. The domain discriminator module can be used to train the feature extractor module to extract domain-invariant and emotion-specific features even though the feature extractor module receives brain signal data of a different domain from the source data.

In detail, the domain discriminator module can make the difference of extracted features small when the feature extractor module receives the source data and the target data. Accordingly, the target data does not need to include reference data, unlike the source data.

The domain discriminator module can receive a value produced when the feature extractor module performs operations. In detail, the domain discriminator module can receive output values of the first and second attention layers of the feature extractor module. The details will be described below.

The domain discriminator module may include a gradient reversal layer (GRL), a fully-connected layer, and a softmax layer.

The gradient reversal layer (GRL) can reverse a gradient in a reverse transmission process. The gradient reversal layer (GRL) can make it difficult for the domain discriminator module to discriminate which one of source data and target data the input data comes from such that the domain discriminator module has difficulty in discriminating source data and target data. That is, the gradient reversal layer can make parameters of the emotion analysis model be updated to increase a discrimination error of the domain discriminator module.

The learning apparatus can update the parameters of the emotion analysis model such that accuracy of the result of expecting emotion recognition is improved and an error of the degree of discrimination by the domain discriminator module is increased in step 350.

The learning apparatus can use a loss function in the process of updating the parameters.

Equation 1 is one of embodiments of a loss function that is used to train the emotion analysis model.

$${L}_{Loss} = {L}_{max - margin} + \text{?} + ( - {L}_{CE} ) \qquad [ Equation 1 ]$$

$$\text{?} \text{indicates text missing or illegible when filed}$$

The loss function may include a total of three items (L_{max-margin}, L_{ds}, L_{CE}) (Loss 1 to Loss 3 in FIG. 6).

The first loss term L_{Loss} relates to a classification loss. The term for the classification loss shows a loss about how well the emotion analysis model recognizes emotions from input brain signal data. The classification loss can be computed through a Contrastive Max-margin function. The purpose of the term for the classification loss is to minimize the probability of other classes and classify samples with high reliability. Equation 1 may be an equation that is used to compute a classification loss.

$${L}_{max - margin} = {\sum }_{k} max ( 0 , 1 + \text{?} ) \qquad [ Equation 2 ]$$

$$\text{?} \text{indicates text missing or illegible when filed}$$

In Equation 2, yₖᵢ means an actual label. In Equation 2, yₖᵢ has a value of −1 when i is the same as yₖᵢ, but has a value of 1 in other cases. In Equation 2, pₖₛ means the probability of belonging to a target emotion class.

The second loss term L_{ds}, is a term for minimizing a distribution shift problem existing in source data. In detail, the second loss term may be considered as a class aware domain similarity loss function (CDF). The second loss term can be computed on the basis of domain similarity output from the classifier module described above.

The second loss term can be formulated as a one-class classification problem. For example, a feature distribution shift of brain signal data obtained from two subjects can be considered. The distribution shift is generated by variability between subjects, and in order to overcome this problem the distribution between features should be minimized. That is, it is possible to minimize a domain shift problem by compressing features of different domains.

Equation 3 and Equation 4 are equations that are used to compute a class aware domain similarity loss. The class aware domain similarity loss can be calculated on the basis of the distances between one sample and the other samples.

$$\text{?} = \text{?} - \frac{\text{?}}{\text{?}} \text{?} \qquad [ Equation 3 ]$$

$$\text{?} \text{indicates text missing or illegible when filed}$$

In Equation 3, p(x_{i,c}) may mean the position of one sample. In Equation 3, p(x_{j,c}) may mean the positions of other samples except for the one sample. In Equation 3, λ may be a hyper-parameter that controls compactness.

$$\text{?} = \frac{\text{?}}{\text{?}} \text{?} \qquad [ Equation 4 ]$$

$$\text{?} \text{indicates text missing or illegible when filed}$$

The third loss term L_{ce} is a term for the domain discriminator module. The third loss term is a term based on cross-entropy. The third loss term maximizes cross-entropy, unlike other terms. According, the domain discriminator module can be trained not to discriminate well which one of source data and target data the input data comes from. This is made possible by the gradient reversal layer (GRL).

Hereafter, an emotion recognition apparatus is described.

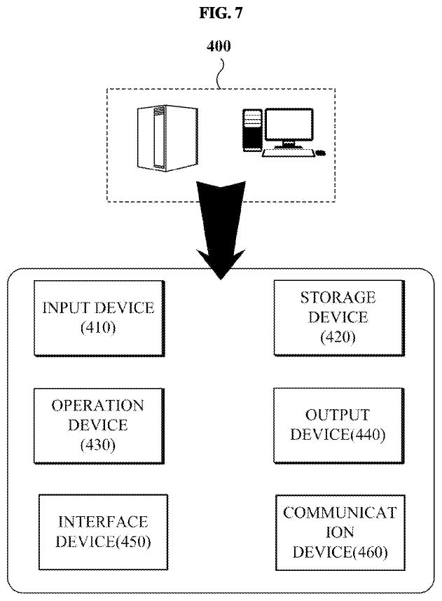

FIG. 7 is one configuration of embodiments of an emotion recognition apparatus.

An emotion recognition apparatus 400 may correspond to the emotion recognition apparatus 100 described with reference to FIG. 1. The emotion recognition apparatus 400 may be an apparatus that performs the method of recognizing an emotion using brain signal data described above.

The emotion recognition apparatus 400 may be an apparatus that perform a method of constructing an emotion analysis model. That is, the emotion recognition apparatus 400 may be the learning apparatus described above.

The emotion recognition apparatus 400 may be implemented physically in various types. For example, the emotion recognition apparatus 400 may have types such as a PC, a laptop, a smart device, a server, or data processing-only chipset.

The emotion recognition apparatus 400 may include an input device 410, a storage device 420, an operation device 430, an output device 440, an interface device 450, and a communication device 460.

The input device 410 may include an interface device (a keyboard, a mouse, a touch screen, etc.) that receives predetermined instructions or data. The input device 410 may include a configuration that receives information through a separate storage device (a USB, a CD, a hard disk, etc.). The input device 410 can receive input data through a separate measurement device or a separate DB. The input device 410 can receive data through wire or wireless communication.

The input device 410 can receive information and a model that are required to perform the method of recognizing an emotion using brain signal data described above. The input device 410 can receive brain signal data. The input device can receive an emotion analysis model.

The input device 410 can receive information and a model that is required to perform the method of constructing an emotion analysis model described above. The input device 410 can receive source data and target data.

The storage device 420 can store information input through the input device 410. The storage device 420 can store information that is created in an operation process of the operation device 430. That is, the storage device 420 may include a memory. The storage device 420 can store the computation result by the operation device 430.

The storage device 420 can store information and a model that are required to perform the method of recognizing an emotion using brain signal data described above. The storage device 420 can store brain signal data. The storage device 420 can store an emotion analysis model.

The storage device 420 can store information and a model that is required to perform the method of constructing an emotion analysis model described above. The storage device 420 can receive source data and target data.

The operation device 430 may be a device such as a processor, an AP, and a program-embedded chip that process data and process predetermined operations. The operation device 430 can generate control signals that control the emotion recognition apparatus 400.

The operation device 430 can perform operations that are required to perform the method of recognizing an emotion using brain signal data described above. The operation device 430 can input brain signal data into the emotion analysis model. The operation device 430 can recognize emotions on the basis of output values of the emotion analysis model.

The operation device 430 can perform operations that are required to perform the method of the emotion analysis model described above.

The operation device 430 can extract the feature of each of data by inputting the source data and the target data into the feature extractor module of the emotion analysis model. The operation device 430 can compute a result of expecting emotion recognition by inputting a feature extracted from the source data into the classifier module of the emotion analysis model. The operation device 430 can input a feature extracted from the target data and a feature extracted from the source data into the domain discriminator module of the emotion analysis model and then can compute the degree of discriminating of the domain discriminator module. The operation device 430 can update the parameters of the emotion analysis model such that accuracy of the result of expecting emotion recognition is improved and an error of the degree of discrimination by the domain discriminator module.

The output device 440 may be a device that output predetermined information. The output device 440 can output an interface required for a data process, input data, an analysis result, etc. The output device 440 may be implemented physically in various types such as a display, a device that outputs documents, etc.

The interface device 450 may be a device that receives predetermined instructions and data from the outside. The interface device 450 can receive brain signal data, source data, target data, and an emotion analysis model from a physically connected input device or external storage device. The interface device 450 can receive control signals for controlling the emotion recognition apparatus 400. The interface device 450 can output an analysis result of the emotion recognition apparatus 400.

The communication device 460 may mean a configuration that receives and transmits predetermined information through a wire or wireless network. The communication device 460 can generate control signals for controlling the emotion recognition apparatus 400. The communication device 460 can transmit an analysis result of the emotion recognition apparatus 400.

The method of recognizing an emotion using brain signal data and the method of constructing an emotion analysis model described above may be implemented into a program (or an application) including a computer-executable algorithm.

The program may be stored and provided in a transitory or non-transitory computer readable medium.

The non-transitory computer readable medium is not a medium that stores data for a short time such as a cache, and a memory, but a medium that can semipermanently store data and can be read out by a device. In detail, the various applications or programs described above may be stored and provided in a non-transitory readable medium such as a CD, a DVC, a hard disk, a Blu-ray disc, a USB, a memory card, a read-only memory (ROM), a programmable read only memory (PROM), an Erasable PROM (EPROM), an Electrically EPROM (EEPROM), or a flash memory.

The transitory readable medium means various RAMs such as a Static RAM (SRAM), a Dynamic RAM (DRAM), a synchronous DRAM (SDRAM), a Double Data Rate SDRAM (DDR SDRAM), an Enhanced SDRAM (ESDRAM), a Synclink DRAM (SLDRAM), and a Direct Rambus RAM (DRRAM).

The embodiments and accompanying drawings only clearly show some of the spirit included in the present disclosure, and it would be apparent that modifications and detailed embodiments that can be easily inferred by those skilled in the art within the spirit included in the specification and drawings are included in the scope of the present disclosure.

What is claimed is:

1. A method of recognizing an emotion using brain signal data, the method comprising:
a step in which an emotion recognition apparatus receives brain signal data;
a step in which the emotion recognition apparatus inputs the brain signal data into an emotion analysis model; and
a step in which the emotion recognition apparatus recognizes an emotion on the basis of an output value of the emotion analysis model,
wherein the emotion analysis model is a model configured to extract a domain-invariant and emotion-specific feature and then analyze an emotion on the basis of the extracted feature.

2. The method of claim 1, wherein the emotion analysis model includes a feature extractor module and a classifier module,
the feature extractor module is a module configured to extract a domain-invariant and emotion-specific feature from the brain signal data, and
the classifier module is a module configured to classify an emotion of a subject when the brain signal data is measured on the basis of the feature extracted by the feature extractor module.

3. The method of claim 2, wherein the feature extractor module sequentially includes a Convolution Neural Network (CNN) layer, a first attention layer, a Long-Short Term Memory (LSTM) layer, and a second attention layer.

4. The method of claim 3, wherein the first attention layer emphasizes a feature for a band having high relevance to emotion recognition in features extracted by the CNN layer, and
the second attention layer emphasizes a feature for a channel having high relevance to emotion recognition in features extracted by the LSTM layer.

5. The method of claim 1, wherein the emotion analysis model is a model constructed by a learning apparatus through following steps (a) to (e) using source data and target data,
(a) a step in which the learning apparatus receives the source data and the target data,
(b) a step in which the learning apparatus extracts a feature of each of data by inputting the source data and the target data into a feature extractor module of the emotion analysis model,
(c) a step in which the learning apparatus computes a result of expecting emotion recognition by inputting a feature extracted from the source data into a classifier module of the emotion analysis model,
(d) a step in which the learning apparatus inputs a feature extracted from the target data and a feature extracted from the source data into a domain discriminator module of the emotion analysis model and then computes the degree of discriminating the source data and the target data of the domain discriminator module, and
(e) a step in which the learning apparatus updates parameters of the emotion analysis model such that accuracy of the result of expecting the emotion recognition is improved and an error of the degree of discrimination by the domain discriminator module is increased,
wherein the source data and the target data are brain signal data belonging to different domains and the source data includes an emotion recognition result that is reference data labeled with correct answers.

6. The method of claim 5, wherein the step (c) is inputting a result emphasizing a feature expected to have high relevance to the emotion recognition in features extracted from the source data through an attention mechanism into the classifier module of the emotion analysis model.

7. The method of claim 5, wherein the step (d) is inputting a result emphasizing a feature expected to have low relevance to the emotion recognition in a feature extracted from the target data and a feature extracted from the source data through an attention mechanism into the domain discriminator module of the emotion analysis model.

8. The method of claim 5, wherein the step (e) is updating parameters using a loss function, and
the loss function includes a term for a classification loss, a term for a class aware domain similarity loss, and a term for a domain discriminator loss.

9. The method of claim 1, wherein the recognizing an emotion includes recognizing an arousal level and a valence level of a subject.

10. An apparatus for recognizing emotions using brain signal data, the apparatus comprising:
an input device configured to receive brain signal data;
an operation device configured to input the brain signal data into an emotion analysis model and recognize an emotion on the basis of an output value of the emotion analysis model; and
a storage device configured to store the emotion analysis model,
wherein the emotion analysis model is a model configured to extract a domain-invariant and emotion-specific feature and then analyze an emotion on the basis of the extracted feature.

11. The apparatus of claim 10, wherein the emotion analysis model includes a feature extractor module and a classifier module,
the feature extractor module is a module configured to extract a domain-invariant and emotion-specific feature from the brain signal data, and
the classifier module is a module configured to classify an emotion of a subject when the brain signal data is measured on the basis of the feature extracted by the feature extractor module.

12. The apparatus of claim 11, wherein the feature extractor module sequentially includes a Convolution Neural Network (CNN) layer, a first attention layer, a Long-Short Term Memory (LSTM) layer, and a second attention layer.

13. The apparatus of claim 12, wherein the first attention layer emphasizes a feature for a band having high relevance to emotion recognition in features extracted by the CNN layer, and
the second attention layer emphasizes a feature for a channel having high relevance to emotion recognition in features extracted by the LSTM layer.

14. The apparatus of claim 10, wherein the emotion analysis model is a model constructed by a learning apparatus through following steps (a) to (e) using source data and target data,
(a) a step in which the learning apparatus receives the source data and the target data,
(b) a step in which the learning apparatus extracts a feature of each of data by inputting the source data and the target data into a feature extractor module of the emotion analysis model,
(c) a step in which the learning apparatus computes a result of expecting emotion recognition by inputting a feature extracted from the source data into a classifier module of the emotion analysis model,
(d) a step in which the learning apparatus inputs a feature extracted from the target data and a feature extracted from the source data into a domain discriminator module of the emotion analysis model and then computes the degree of discrimination by the domain discriminator module, and
(e) a step in which the learning apparatus updates parameters of the emotion analysis model such that accuracy of the result of expecting the emotion recognition is improved and an error of the degree of discrimination by the domain discriminator module is increased,
wherein the source data and the target data are brain signal data belonging to different domains and the source data includes an emotion recognition result that is reference data labeled with correct answers.

15. The apparatus of claim 14, wherein the step (c) is inputting a result emphasizing a feature expected to have high relevance to emotion recognition in features extracted from the source data through an attention mechanism into a classifier module of the emotion analysis model.

16. The apparatus of claim 14, wherein the step (d) is inputting a result emphasizing a feature expected to have low relevance to the emotion recognition in a feature extracted from the target data and a feature extracted from the source data through an attention mechanism into the domain discriminator module of the emotion analysis model.

17. The apparatus of claim 14, wherein the step (e) is updating parameters using a loss function, and
the loss function includes a term for a classification loss, a term for a class aware domain similarity loss, and a term for a domain discriminator loss.

18. The apparatus of claim 10, wherein the recognizing an emotion includes recognizing an arousal level and a valence level of a subject.

19. A method of constructing the emotion analysis model by means of a learning apparatus, the method comprising:
(a) a step in which the learning apparatus receives source data and target data;
(b) a step in which the learning apparatus extracts a feature of each of data by inputting the source data and the target data into a feature extractor module of the emotion analysis model;
(c) a step in which the learning apparatus computes a result of expecting emotion recognition by inputting a feature extracted from the source data into a classifier module of the emotion analysis model;
(d) a step in which the learning apparatus inputs a feature extracted from the target data and a feature extracted from the source data into a domain discriminator module of the emotion analysis model and then computes the degree of discrimination by the domain discriminator module; and
(e) a step in which the learning apparatus updates parameters of the emotion analysis model such that accuracy of the result of expecting the emotion recognition is improved and an error of the degree of discrimination by the domain discriminator module is increased,
wherein the source data and the target data are brain signal data belonging to different domains and the source data includes an emotion recognition result that is reference data.

20. The method of claim 19, wherein the step (c) is inputting a result emphasizing a feature expected to have high relevance to emotion recognition in features extracted from the source data through an attention mechanism into the classifier module of the emotion analysis model, and
the step (d) is inputting a result emphasizing a feature expected to have low relevance to the emotion recognition in a feature extracted from the target data and a feature extracted from the source data through an attention mechanism into the domain discriminator module of the emotion analysis model.
